(19) United States
(12) Patent Application Publication
(10) Pub. No.: US 2003/0114745 A1
(43) Pub. Date: Jun. 19, 2003

(54) Biomagnetic field measuring system

(76) Inventors: Yoshiaki Amano, Tama (JP); Mitsuru Oonuma, Tokyo (JP); Taro Ishikawa, Inagi (JP); Kazunori Yanagisawa, Higashiyamato (JP); Hitoshi Sasabuchi, Mito (JP); Shoji Kondo, Hitachinaka (JP)

Correspondence Address: Mattingly, Stanger & Malur, P.C., Suite 370, 1800 Diagonal Road, Alexandria, VA, 22314, US

(21) Appl. No.: 10/293,429

(22) Filed: Nov. 14, 2002

(30) Foreign Application Priority Data
Dec. 19, 2001 (JP) 2001-386535

(51) Int. Cl. A61B 5/05

(52) U.S. Cl. 600/409

(57) ABSTRACT

In a magnetically shielded room, a cryostat that is filled up with liquid He serving as a coolant and that houses magnetic sensors is installed. A bed for laying down a suspect to be inspected thereon is disposed on a floor surface under the cryostat. After the subject to be inspected is laid down on the bed, the bed is positioned by the inspector. Near the door of the shielded room, an intra-shielded-room operation input device for the inspector to conduct operation on a system control device is provided. A through hole is opened through a part of the wall, and a projector is disposed outside the through hole. An inspection result and so on are projected from the projector onto a wall surface of a partition wall, and used for head positioning and so on.

### BACKGROUND OF THE INVENTION

The present invention relates to a biomagnetic field measuring system, and in particular to a biomagnetic field measuring system suitable for measurement of a biomagnetic field generated from a current within a living body, such as nerval activity of a brain of a living body or cardiac muscle activity of a heart.

As conventional techniques concerning the biomagnetic field measurement, techniques described in, for example, JP-A-4-319334 and JP-A-5-146416 are known. These conventional techniques includes the steps of measuring distribution of a weak magnetic field generated from a living body by using a superconductive quantum interference device (SQUID), estimating locations of active currents within a living body, and imaging the distribution.

Such an apparatus for conducting biomagnetic measurement must detect extremely weak magnetism generated from a living body. In order to avoid the influence of terrestrial magnetism and the influence of magnetic fields or electromagnetic fields generated from electronic devices that exist in the periphery, the apparatus for conducting biomagnetic measurement is typically installed in a room wholly shielded by wall surfaces that are formed of a magnetic material such as permalloy. Furthermore, in order to eliminate the magnetic field or electromagnetic field generated from a device installed in a room, all devices relating to sensors of a control section and an inspection information display section of the apparatus, except a sensor for detecting magnetism from a subject to be inspected, are installed outside the room.

All of the above described conventional techniques relate to the operation principle of a biomagnetic imaging apparatus, and technical problems and solving means in implementation are not disclosed. Furthermore, the above described conventional techniques aim at detecting magnetism caused by a bioactivity current generated within the brain, and concrete disclosure concerning other regions is not effected.

In using a biomagnetic measuring apparatus according to the above described conventional techniques, an inspector guides a subject to be inspected (a patient) in the room magnetically shielded as described above, places the subject to be inspected on a bed disposed under an apparatus installed in the room, and positions the subject to be inspected so as to make the inspection location of the subject to be inspected coincide with a location of a sensor of the apparatus. Thereafter, the inspector goes out of the room once, makes the biomagnetic measuring apparatus start provisional measurement and display a result of the measurement, and makes sure whether the positioning of the subject to be inspected has been conducted properly, on the basis of the result. If the positioning of the subject to be inspected has not been conducted properly, then the inspector enters the room again and conducts the work of re-setting the position of the subject to be inspected.

In the biomagnetic measuring apparatus according to the conventional techniques, it is necessary to conduct the work of positioning the subject to be inspected so as to make the inspection location of the subject to be inspected coincide with the location of the sensor of the apparatus, while watching the screen of a display device installed outside the room in which the apparatus is installed. Until the subject to be inspected can be positioned properly, therefore, the inspector must come and go between the inside and the outside of the room many times. This results in a problem that the amount of work of the inspector is large and a lot of time is required.

Furthermore, in the conventional techniques, the subject to be inspected is left in the inspection room closed up tightly, while the positioning of the subject to be inspected is being conducted and during a time period (approximately 5 minutes at most) between the start and end of the actual inspection. This results in a problem that the subject to be inspected becomes uneasy.

### SUMMARY OF THE INVENTION

An object of the present invention is to provide a biomagnetic measuring system that solves the problems of the conventional techniques, facilitates the measurement of magnetic field intensities at a plurality of arbitrary measurement locations of a living body, and facilitates user's operation.

Another object of the present invention is to provide a biomagnetic measuring system that can make the inspector conduct the work efficiently and yet that does not make the subject to be inspected uneasy.

In accordance with the present invention, the object is achieved by a biomagnetic measuring system for measuring a biomagnetic field, including an image projection section disposed on a wall surface in a magnetically shielded room, and a projector for projecting an image onto the image projection section, the projector being disposed outside the magnetically shielded room, flux of light from the projector being projected onto the image projection section through a duct disposed so as to pass through a wall surface of the magnetically shielded room.

Other objects, features and advantages of the invention will become apparent from the following description of the embodiments of the invention taken in conjunction with the accompanying drawings.

### BRIEF DESCRIPTION OF THE DRAWINGS

FIG. 1 is a block diagram showing a configuration example of a biomagnetic measuring system according to an embodiment of the present invention;

FIG. 2 is an oblique view showing an arrangement state of devices in a magnetically shielded room;

FIG. 3 is a diagram showing a state of eyes of a subject to be inspected;

FIGS. 4A and 4B are diagrams showing examples of a state in which a subject to be inspected is laid down;

FIG. 5 is an oblique view showing a configuration of a duct;

FIGS. 6A to 6C are diagrams showing display states of inspection information projected through a duct;

FIG. 7 is a flow chart showing operation of an inspector in the case where inspection of a subject to be inspected is conducted by using an embodiment of the present invention;

FIG. 8 is a diagram showing a configuration of operation buttons of an intra-shielded-room operation input device provided in a shielded room;

FIGS. 9A to 9F are diagrams showing some examples of a video image shown to a subject to be inspected in the midst of a measurement; and

FIGS. 10A and 10B are block diagrams showing a configuration example of a biomagnetic measuring system according to another embodiment of the present invention.

### DESCRIPTION OF THE EMBODIMENTS

Hereafter, embodiments of a biomagnetic measuring system according to the present invention will be described in detail with reference to the drawings.

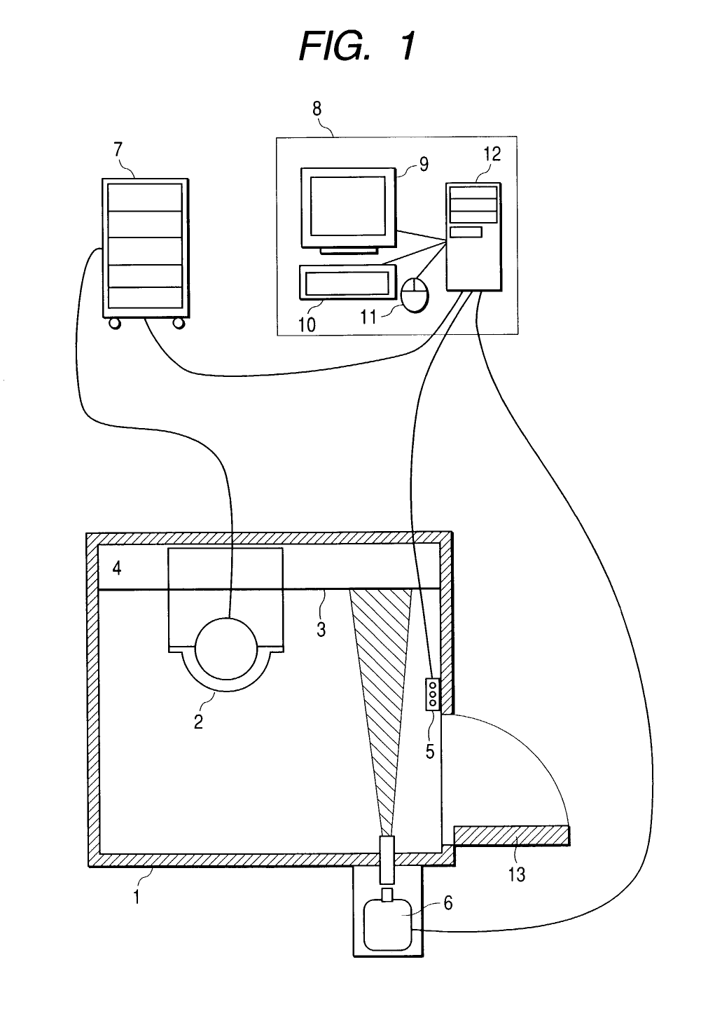

FIG. 1 is a block diagram showing a configuration example of a biomagnetic measuring system according to an embodiment of the present invention. First, an outline of the system configuration will now be described with reference to FIG. 1. In FIG. 1, reference numeral 1 denotes a magnetically shielded room, 2 a cryostat, 3 a partition wall, 4 a side room, 5 an intra-shielded room operation input device, 6 a projector, 7 a measurement control unit, 8 a system control device, 9 a display, 10 a keyboard, 11 a mouse, 12 an information processing device, and 13 a door.

For conducting biomagnetic measurement, it is necessary to avoid the influence of terrestrial magnetism and the influence of magnetic fields or electromagnetic fields generated from electronic devices that exist in the periphery, as described in “BACKGROUND OF THE INVENTION.” In a biomagnetic measuring system according to an embodiment of the present invention, therefore, a cryostat 2 filled up with liquid He serving as a coolant is installed in a magnetically shielded room 1 surrounded by front, rear, right, left, top and bottom walls and the door 13 as shown in FIG. 1. Each of the front, rear, right, left, top and bottom walls is approximately 10 cm in thickness. And a magnetic material such as permalloy having a thickness of approximately 2 mm is stuck to the inside surface and outside surface of each of the front, rear, right, left, top and bottom walls. In the same way, the door 13 is formed of permalloy or the like. An inspector goes in and out through the door 13. (FIG. 1 is a top view of the shielded room, and details of the inside will be described later.) A plurality of magnetic sensors are housed in the cryostat 2. Each magnetic sensor includes a superconducting quantum interference device (SQUID) and a detection coil connected to the SQUID. The magnetically shielded room 1 has a floor area of approximately 2 m by 2 m. The partition wall 3 is provided on the side on which the cryostat 2 is installed. In the side room 4 formed by the partition wall 3 and the wall of the shielded room 1, a tank of liquid He to be supplied to the cryostat 2 and a support pillar of the cryostat 2 are disposed. The tank and the support pillar are not illustrated.

Furthermore, although not shown in FIG. 1, a bed for laying down a suspect to be inspected thereon is disposed on a floor surface of the shielded room 1 under the cryostat 2. The bed can be moved upward, downward, leftward, rightward, forward and backward. After the subject to be inspected is laid down on the bed, the bed is positioned by the inspector. As a result, a region to be inspected is opposed to a magnetic sensor that is provided on a lower part of the cryostat 2. In addition, near the door 13 of the shielded room 1 and near an image projection section described later, the intra-shielded-room operation input device 5 for the inspector to conduct operation on the system control device 8 described later is provided. Although described in detail later, a through hole is opened through a part of the wall of the magnetically shielded room 1, and a projector 6 is disposed outside the through hole. A duct described later is disposed in the through hole. Projected light from the projector 6 is passed through the duct to project an inspection result and so on the wall surface of the partition wall 3.

The projector 6 installed outside the magnetically shielded room 1 is a projector using liquid crystal or the like. The projector 6 projects a video signal sent from the information processing device 12 serving as a main body of the system control device 8. By installing the projector 6 outside the magnetically shielded room 1, it is possible to prevent the magnetic flux generated from the projector itself from intruding into the shielded room 1.

As already described, it is necessary to make the inside of the shielded room an environment in which magnetism functioning as noise is reduced to the utmost. As for the illumination in the room as well, a light source using electricity, such as an electric bulb, cannot be used. Therefore, illumination is conducted by using optical fiber or the like, and a high intensity of illumination cannot be obtained. In the embodiment of the present invention, a video signal or the like is projected from the projector 6 installed outside into the shielded room 1 as described above. Therefore, reflected light from a resultant image projection section on the wall surface can increase the intensity of illumination in the room.

The dedicated measurement control unit 7 for controlling the cryostat 2 having a magnetic sensor installed in the shielded room, and the system control device 8 for controlling the whole system are installed outside the magnetically shielded room 1. The system control device 8 includes the display 9 for displaying an inspection result and so on, an input devices such as the keyboard 10 and the mouse 11, and the information processing device 12, such as a personal computer, serving as the main body of the control device. In response to an inspector's order input from an input device such as the keyboard 10 and the mouse 11, or the intra-shielded-room operation input device 5, the information processing device 12 conducts control of start of inspection on the subject to be inspected, processing of collected data of the inspection result, and display of the processing result on the display 9. The information processing device 12 also controls projection of the inspection result or video information shown to the subject to be inspected from the projector 6.

In the biomagnetic measuring system having the above described configuration according to an embodiment of the present invention, outputs of a plurality of magnetic sensors in the cryostat 2 are output as voltages that have specific relations with intensities (which can be regarded as magnetic flux densities) of biomagnetic fields generated from the subject to be inspected and detected by detection coils, input to the information processing device 12 via the measurement control unit 7, and processed therein. Details of how to take out outputs of the magnetic sensors and the processing method are described in, for example, JP-A-11-104094. They are not essentials of the present invention, and description thereof will be omitted.

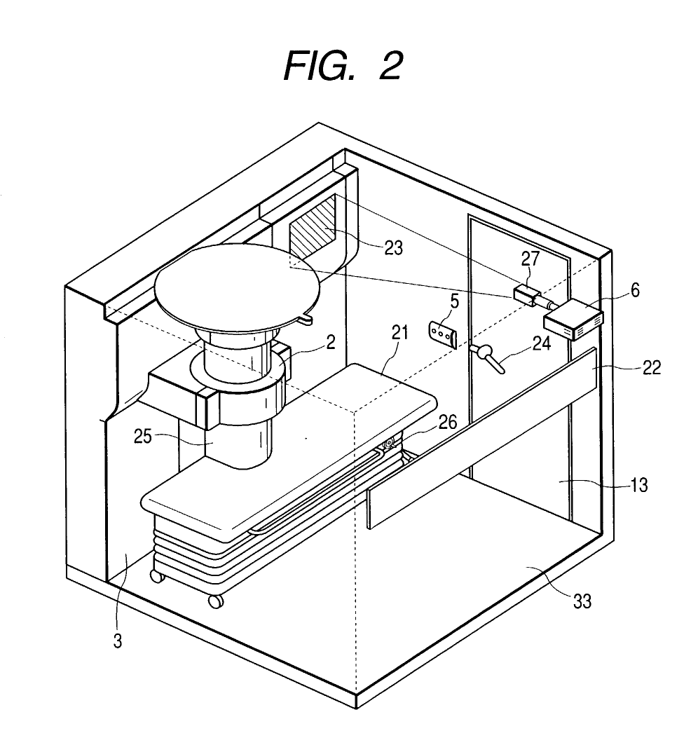

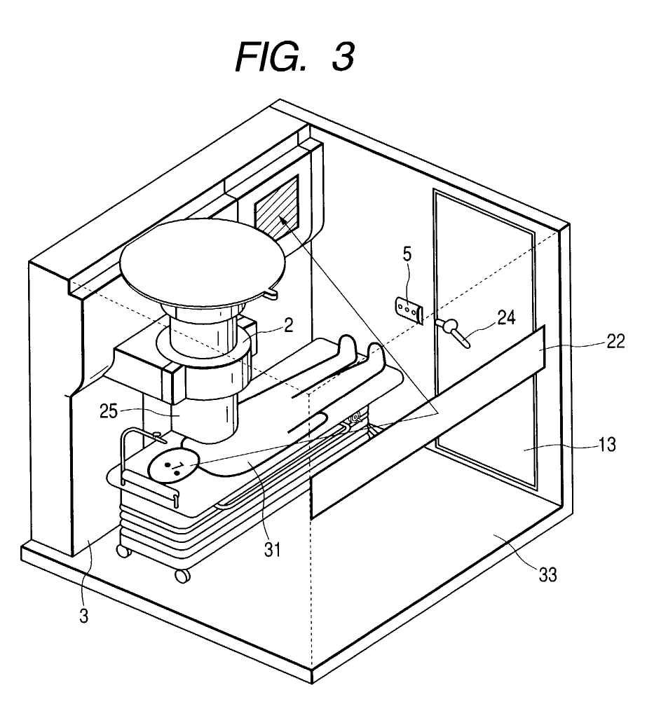

FIG. 2 is an oblique view showing an arrangement state of devices in a magnetically shielded room. FIG. 3 is a diagram showing a state of eyes of a subject to be inspected. In FIGS. 2 and 3, reference numerals 21 denotes a bed, 22 a mirror, 23 an image projection section, 24 a door knob, 25 a sensor housing section, 26 a bed positioning lever, 27 a duct, and 31 a subject to be inspected. Other reference numerals are the same as those of FIG. 1.

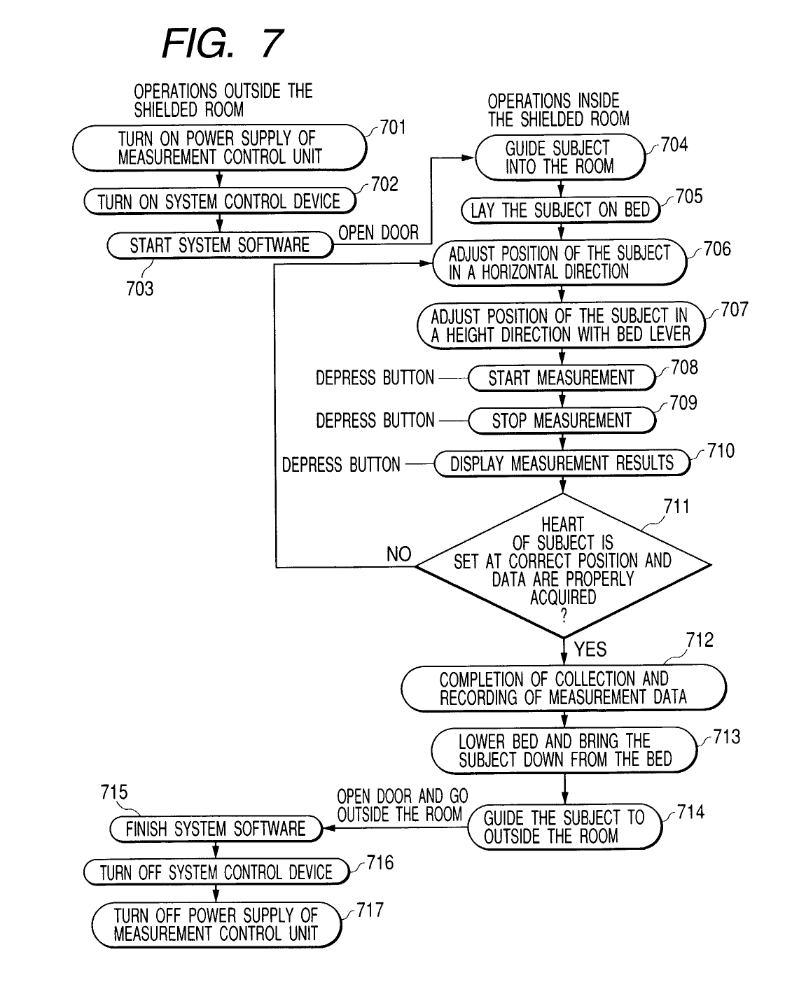

As shown in FIG. 2, the lower end of the cryostat 2 installed in the magnetically shielded room 1 and described above is used as the sensor housing section 25. The bed 21 is installed between the sensor housing section 25 and a floor surface 33 of the shielded room 1. The bed 21 has a bed positioning level 26. With the subject to be inspected on the bed, position of the head 21 in the front and rear direction, the left and right direction, and in the height direction can be conducted. Since the inspector manually operates the bed positioning lever 26, the operator needs to operate it in a squatting posture. As described later with respect to operation of the inspector with reference to FIG. 7, it becomes necessary for the inspector to watch the image projection section 23 while maintaining the posture for operating the bed positioning lever 26.

As understood from FIG. 2 as well, the bed 21 is disposed on the partition wall 3 side. A long and narrow mirror 22 is disposed on a wall surface of the shielded room opposed to the partition wall 3. A space between the bed 21 and the wall surface of the shielded room opposed to the partition wall 3 becomes a work space of the inspector. The door 13 having the door knob 24 is provided through a wall of the shielded room that forms a side face of the space. In addition, the intra-shielded-room operation input device 5 is provided on the wall surface of the shielded room 1 near the door 13 and in the position of hands of the inspector.

It is desirable that the intra-shielded-room operation input device 5 is disposed in such a position that the operator can easily reach while maintaining the posture for squatting down and operating the bed positioning lever 26, and yet in a position that is not too low so that the operator may easily reach in a standing posture. In other words, it is desirable that the intra-shielded-room operation input device 5 is in such a position that the operator can easily reach while maintaining the posture for squatting down and operating the bed positioning lever 26, and yet in such a height that the operator can easily operate it in a standing posture immediately after the operator has stepped into the shielded room through the entrance.

The height dimension of the intra-shielded-room operation input device 5 from the floor surface 33 of the intra-shielded-room operation input device 5 will now be considered. It is now supposed that central portions of a plurality of operation buttons disposed in the height direction and the lateral direction are Hi (i=1, 2, . . . ) in height from the floor surface 33. On the other hand, a height of hands in a posture of a person who drops an arm, bends an elbow, and makes the arm beyond the elbow nearly parallel to the ground is the height facilitating the operation. On the basis of a 5% tile value of the height of a hand in a posture of a Japanese woman raising her arm beyond the elbow by 10 degrees and a 95% tile value of the height of a hand in a posture of a Westerner man lowering his arm beyond the elbow by 10 degrees, it is suitable that the height Hi satisfies the relation 970 mm<Hi<1130 mm.

By the way, the 5% tile value of stature of the Japanese women is 1495 mm, and the 95% tile value of stature of the Westerner men is 1880 mm.

If the height dimension of the intra-shielded-room operation input device 5 from the floor surface 33 is set in the above described range, then each of a person having a stature of the 5% tile of the Japanese women stature and a person having a stature of the 95% tile of the Westerner men stature can easily conduct operation of the intra-shielded-room operation input device 5 by stretching a hand upward in such a posture that the person is squatting down before the lever 26 with a knee of a leg drawn up.

By setting the height Hi into the range of 970 mm<Hi<1130 mm, therefore, the inspector can easily operate the intra-shielded-room operation input device 5 no matter whether the inspector is in a standing posture or in a squatting posture in order to operate the lever 26.

It is desirable that the height between the floor surface 33 and the lower end of the duct 27 is at least 1780 mm in Japan and at least 1880 mm in Europe and America. These numerical values are based on the stature of 95% tile of men in respective areas, By the way, the 95% tile stature is 1780 mm in Japan, and 1880 mm in Europe and America. By setting the duct 27 to the above described height, the inspector becomes hard to strike the duct 27 on the head and safety can be ensured. In addition, it becomes possible to prevent that a flux of light is intercepted by the head of the inspector and a part or the whole of a projected image is missing.

Therefore, it is suitable to set the height between the floor surface 33 and the lower end of the duct 27 equal to at least 1880 mm (and less than the height of the ceiling). By doing so, it is possible to prevent the video image projected from the duct 27 from being intercepted even if a person crosses the duct 27 in front of it.

The projector 6 described with reference to FIG. 1 is disposed so as to pass projected light through the duct 27 inserted in the through hole, which is formed through an upper part of the shielded room wall having the mirror 22. Therefore, the projector 6 is disposed so as to position its projection lens in an opening portion of the duct 27 located outside the shielded room 1. A video image projected from the projector 6 is projected on the image projection section 23, which is located in an upper corner of the shielded room 1. The video image projected on the image projection section 23 is referred to, for the purpose of positioning the bed when the inspector executes the inspection, as described later. Furthermore, when the subject 31 to be inspected stays alone in the shielded room, the video image projected on the image projection section 23 can be used to know the progress of the inspection.

Information to be known by the inspector is typically only graphical information of the inspection result. If only this information is needed, the size of the image projection section 23 needs only be approximately 15 inches.

The bed positioning lever 26 is located in front of the image projection section 23, i.e., located between the image projection section 23 and the wall opposed to the image projection section 23. And the image projection section 23 needs to be disposed at a such a location that it can be easily watched from the location where the bed positioning lever 26 is operated. In this case, “such a location that it can be easily watched” means such a location that the operator can watch the screen without strain by moving the head and moving eyes at the lever operation location. The range in which the operator can watch the screen without strain by moving the head and moving eyes is approximately 40 degrees on each of the left and right sides with respect to the front of a human body.

It is considered that the head of the inspector operating the lever 26 is nearly in the location of the lever 26. When the operator is rightly opposed to the partition wall 3, therefore, the image projection section 23 should be positioned in the range of 40 degrees on each of the left and right sides, i.e., in the range of a total of 80 degrees, from the location of the head positioning lever 26. In other words, the image projection section 23 should be positioned in the above described range, considering only the horizontal component seen from the right above. By positioning the image projection section 23 in such a range, the operator can watch the screen by moving the head and eyes without strain. Even in the range of 40 degrees on each of the left and right sides, it is necessary to avoid to the utmost that an image is projected over a plurality of walls and that the image projection section 23 extends over the cryostat 2.

The projection surface (which is parallel to the plane of the partition wall 3) need not be perpendicular to the flux of light. Because an image adjustment function of the projector 6 can correct the distortion in the vertical direction and the distortion in the horizontal direction of the projection image of the image projection section 23 according to the location where the inspector operates the lever 26.

The relation between the eyes of the subject to be inspected lying on the bed 21 in the shielded room 1 having the device arrangement described above and the mirror 22 will now be described with reference to FIG. 3. FIG. 3 shows the state in the shielded room 1 in the same way as FIG. 2. In FIG. 3, however, the projector 6 is omitted and the state of the subject 31 to be inspected lying on the bed 21 is shown.

As shown in FIG. 3, the subject 31 to be inspected lies with the head located before the wall opposed to the wall of the shielded room 1 having the door 13. In the example of FIG. 3, the sensor housing section 25 of the cryostat 2 is positioned over the breast of the subject 31 to be inspected. If in such a state eyes of the subject 31 to be inspected are directed to mirror 22, then the subject 31 to be inspected can look at the video image projected and displayed on the image projection section 23 via the mirror 22. Since the the subject to be inspected thus look at the video image projected and displayed on the image projection section 23 via the mirror 22, the height position of the mirror 22 is preferably located at nearly middle between the height of eyes of the subject to be inspected and the height of the image projection section 23. Furthermore, the height dimension of the mirror 22 itself is set equal to such a dimension that the subject 31 to be inspected can look at the whole of the image projection section 23 even if the position (height) of the eyes of the subject 31 to be inspected is slightly changed, on the basis of the position of the eyes of the subject 31 to be inspected, which is changed in the adjustable range of the bed 21, and the dimension of the image projection section in the height direction.

The disposition location of the mirror 22 in the width direction and the mirror itself can be determined on the basis of the position of the eyes of the subject 31 to be inspected and the position of the image projection section 23 in the same way as the case of the height position and the height dimension described above. The width direction need not be the whole width of the shielded room 1 as shown in FIG. 3. Depending upon the purpose of the invention, however, it becomes necessary to invert the direction in which the subject 31 to be inspected is laid as described later. Therefore, it is possible that the position of the eyes of the subject 31 to be inspected is greatly changed. Accordingly, the width dimension of the mirror 22 is desired to be long to some extent. Furthermore, since the mirror 22 reflects the inside of the room other than the image projection section 23, it brings about an effect of making the space in the room look wide to the subject to be inspected. In this case, it is effective to make the height dimension and the width dimension of the mirror 22 larger.

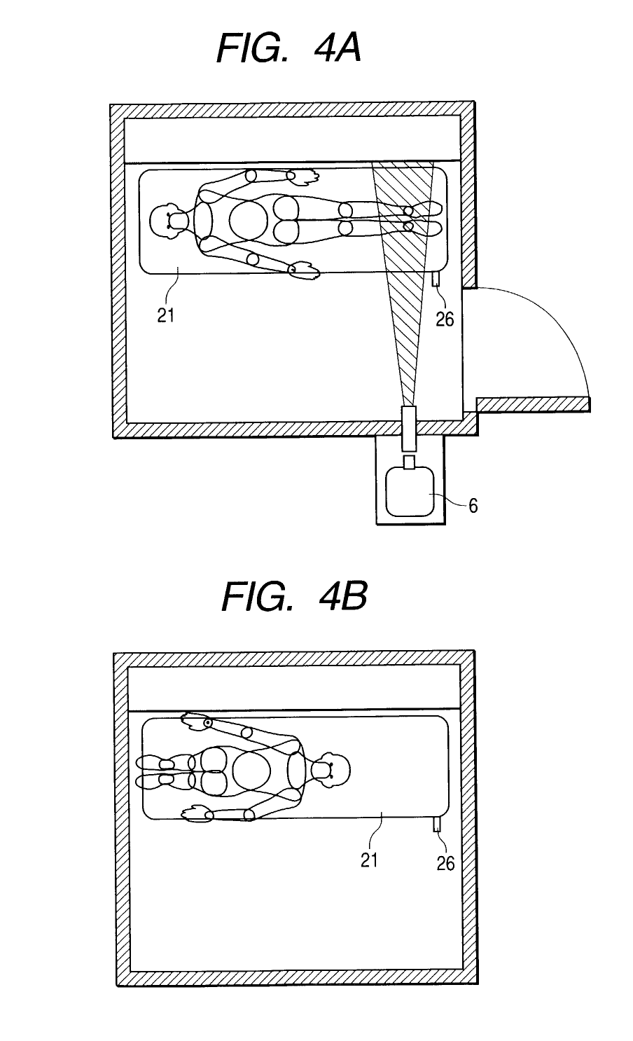

FIGS. 4A and 4B are diagrams showing examples of a state in which a subject to be inspected is laid down. FIG. 4A shows a typical state in which inspection of the heart of the subject 31 to be inspected is conducted. This state is the state of the subject to be inspected described with reference to FIG. 3. Typically, inspection is conducted in such a state. As a matter of course, the head position of the subject 31 to be inspected is located in a required position of the bed 21 in the lengthwise direction according to a region to be inspected of the subject 31 to be inspected. The state shown in FIG. 4B is a state in which inspection of a foetus of the subject 31 to be inspected is conducted. In this state, the head of the subject 31 to be inspected is placed in a direction opposite to that of FIG. 4A, and the subject 31 to be inspected is laid with her knees bent. A sensor is positioned over the abdomen of the subject 31 to be inspected having the foetus. If the subject 31 to be inspected directs eyes to the direction of the mirror 22 disposed as described with reference to FIG. 3, then the subject 31 to be inspected can look at the video image projected on the image projection section 23 even in the state shown in FIG. 4B.

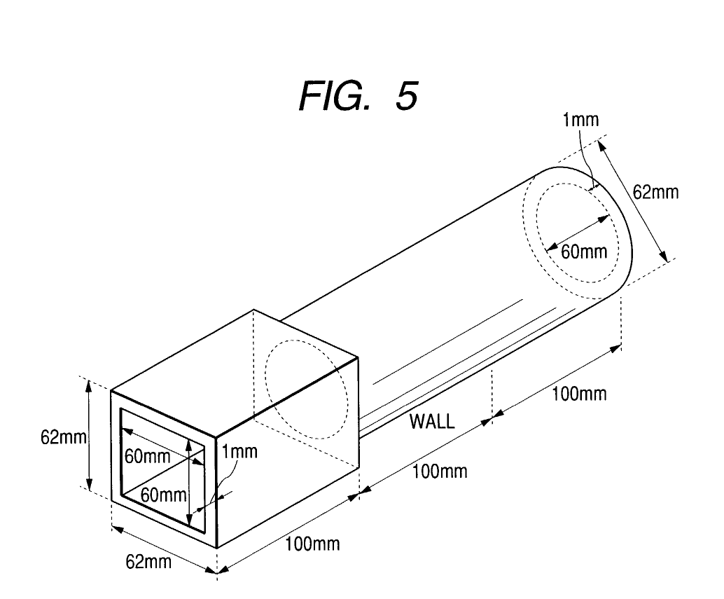

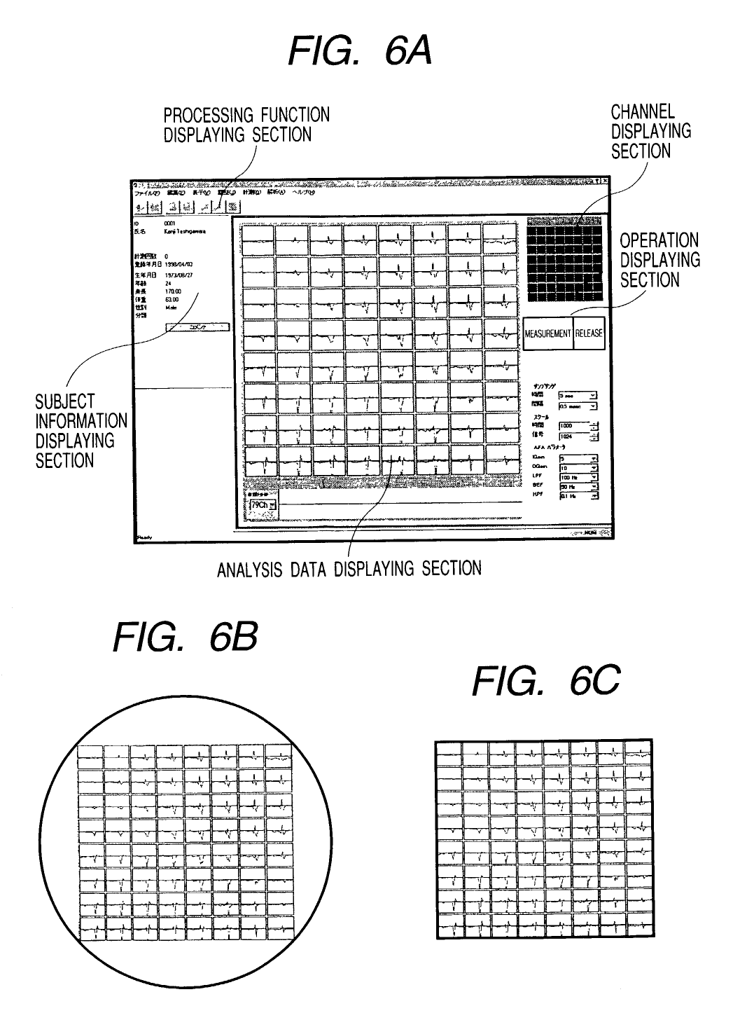

FIG. 5 is an oblique view showing a configuration of the duct 27. FIGS. 6A to 6C are diagrams showing display states of inspection information projected through the duct. The relation between the duct shape and displayed video images will now be described.

As shown in FIG. 5, the duct 27 is formed of two pipes: an inner pipe and an outer pipe. The two pipes are formed of a magnetic material such as permalloy having a thickness of approximately 1 mm. The outer pipe has a shape of a trapezium and a length of approximately 100 mm. The inner pipe has a shape of a circle and a length of approximately 200 mm. Therefore, the total length becomes approximately 300 mm. It is a value experimentally conformed as a minimum length capable of making the magnetic field intruding into the inside through the hole of the wall of the shielded room 1 sufficiently small. The circular portion is approximately 60 mm in inside diameter, approximately 62 mm in outside diameter, and approximately 200 mm in length. The trapezium portion coupled to the circular portion is approximately 62 mm by 62 mm in outer side, approximately 60 mm by 60 mm in opening, and approximately 100 mm in length.

The duct 27 having the above described configuration is attached to the hole formed through the wall of the shielded room 1 with the circular portion disposed on the room outside side and the trapezium portion disposed on the room inside side. The thickness of the wall of the shielded room 1 is approximately 100 mm as already described. As shown in FIG. 5, therefore, a portion of the duct 27 having a length of 100 mm from a coupling position between the circular portion and the trapezium portion is disposed within the thickness of the wall of the shielded room 1. A portion of the circular portion having a length of 100 mm from the tip of the circular portion is exposed to the outside of the shielded room 1. The trapezium portion is attached to be exposed to the inside of the shielded room 1. In the duct 27, the circular portion passes through the wall. In the present invention, however, it is also possible to provide flanges on the circular portion and the trapezium portion, form portions each having a length of 100 mm independently, and attach them to edges of the hole formed through the wall by using the flanges. Or the portions may be attached to permalloy of the wall surface by means of soldering without providing the flanges.

Display of inspection information projected through the duct 27 will now be described with reference to FIGS. 6A to 6C.

An example of inspection information shown in FIG. 6A is typically an example of an image displayed on the display 9 described with reference to FIG. 1. Besides an analysis data display section for displaying analysis data, all information required for inspection, such as a subject information display section, a processing function display section, a channel display section, and an operation display section, is included. In the analysis data display section in the example shown in FIG. 6A, results obtained by conducting processing on detection signals respectively of 64 magnetic sensors described earlier and arranged in an 8 by 8 form are shown.

However, information required for the inspector to enter the shielded room 1 and precisely determining the position of the subject to be inspected is only information as to whether inspection is being conducted normally. Therefore, the section required to be referred to when positioning the head is only the analysis data display section. In the embodiment of the present invention, the projector 6 displays only the analysis data display section in the shielded room 1 for the inspector with an area as large as possible. However, the present invention is not limited to this, but all images shown in FIG. 6A may also be displayed. In this case, it becomes possible for the inspector to conduct all inspection processing within the shielded room by bringing the input devices such as the mouse into the shielded room 1.

For displaying only the analysis data display section with an area as large as possible, the shape of the duct 27 described with reference to FIG. 5 is effective. This will now be described.

The simpler the shape of the duct 27 has, the better from the viewpoint of cost. For example, the duct 27 is desired to be cylindrical. If the duct 27 is formed in a cylindrical shape and it is attempted to project only the analysis data display section as shown in FIG. 6A from the projector disposed outside the shielded room 1 onto the image projection section 23 in the shielded room 1, then the size of the analysis data display section in the diagonal direction is limited by the inside diameter of the duct 27 as shown in FIG. 6B, because the analysis data display section is formed in a rectangular form. The area of the analysis data display section projected on the image projection section 23 cannot be made so large, and a wasteful portion occurs.

In the case of the shape of the duct 27 described with reference to FIG. 5, the trapezium portion is positioned inside the shielded room 1. The projected flux of light from the projector 6 disposed in the inlet of the circular portion disposed outside the shielded room 1 arrives at the trapezium portion while it is being expanded. At the outlet of the trapezium portion, outer edges of the analysis data display section of the image information are expanded to the size and shape of the outlet and projected. As a result, the analysis data display section projected onto the image projection section 23 is displayed as shown in FIG. 6C. As understood from the shape of the duct 27, the diagonal size of the analysis data display section displayed on the image projection section 23 shown in FIG. 6C can be made equal to the diagonal size of a trapezium circumscribed about the circle shown in FIG. 6B. Thus, the size of the the analysis data display section in the diagonal direction can be made markedly greater as compared with the case of FIG. 6B. If it is sufficient to obtain a screen as large as that of the case shown in FIG. 6B, the whole of the duct 27 can be formed so as to have a sectional shape of a trapezium that is smaller in section than the size of the trapezium portion, and consequently magnetism intruding into the shielded room via the duct can be reduced.

In the present embodiment of the present invention, the analysis data can be displayed in the shielded room 1 as described above. In the shielded room, therefore, the inspector can position the bed while watching the analysis data, precisely determine the position of the subject to be inspected, and subsequently conduct real inspection. Therefore, it becomes possible to prevent the inspector from coming and going between the inside and the outside of the shielded room 1 many times.

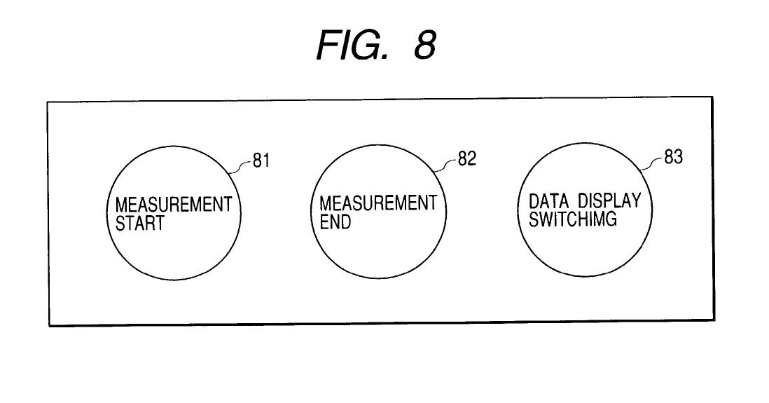

FIG. 7 is a flow chart showing operation of the inspector in the case where inspection of the subject to be inspected is conducted by using the embodiment of the present invention. FIG. 8 is a diagram showing a configuration of operation buttons of the intra-shielded-room operation input device 5 provided in the shielded room 1. First, the operation buttons of the intra-shielded-room operation input device 5 will now be described.

As shown in FIG. 8, the intra-shielded-room operation input device 5 includes a measurement start button 81, a measurement end button 82, and a data display switching button 83. The measurement start button 81 is operated when starting a provisional measurement and a real measurement. The provisional measurement is conducted for checking to determine whether a region to be inspected of the subject to be inspected is properly opposed to a sensor when determining the position of the subject to be inspected. The measurement end button 82 is operated when finishing the above described measurement and displaying the analysis data. By using the data display switching button 83, it is possible to cyclically effect a selection and switching to determine whether the analysis data described above should be displayed or the progress of the inspection and the environmental information should be displayed for the subject to be inspected.

Operation of the inspector conducted when effecting an inspection on the subject to be inspected will now be described with reference to the flow chart of FIG. 7.

(1) Prior to the inspection start, the inspector first turns on power supply of the measurement control unit 7 and the system control device 8 and starts system software. At this time, the inspector inputs the information of the inspector to be inspected and necessary processing functions as occasion demands (steps 701 to 703).

(2) The inspector then opens the door of the magnetically shielded room 1, guided the subject to be inspected (patient) into the room, lay the subject to be inspected down on the bed 21, and adjust the position of the bed in the horizontal direction and the height direction. The work of the inspector heretofore described is conducted while confirming the mutual position relation between the sensor housing section 25 of the cryostat 2 and the region to be inspected of the subject to be inspected by watching them. During that time, the progress of the inspection and the environmental information can be displayed on the image projection section 23 and shown to the subject to be inspected (steps 704 to 707).

(3) The inspector then depresses the measurement start button 81 to start a measurement, and depresses the measurement button 82 to stop the measurement. In addition, the inspector operates the data display switching button 83 to display measurement results on the image projection section 23 (steps 708 to 710).

(4) By watching the picture of the measurement results displayed on the image projection section 23, the operator (inspector) determines whether the region to be inspected (heart in the illustrated example) of the subject to be inspected is set in the proper position and data are acquired properly. If data have not been acquired properly, the operator repeats the work of adjusting the position of the bed beginning with the step 706 using the bed positioning lever 26 and the measurement. At this time, it is desirable that the image projection section 23 is in such a position that it can be easily watched in a posture for operating the bed positioning lever 26, as described above (step 711).

(5) If the data have been acquired properly in the decision at the step 711, then the inspector collects and record measurement data, and finishes the measurement work (step 712).

(6) Thereafter, the inspector lowers the bed 21, brings the subject to be inspected down from the bed, opens the door 13, guides the subject to be inspected to the outside of the room, and releases the subject to be inspected (steps 713 and 714).

(7) Finally, the inspector finishes the system software, turns off the power supply of the system control device 8 and the measurement control unit 7, and finishes all inspection works (steps 715 to 717).

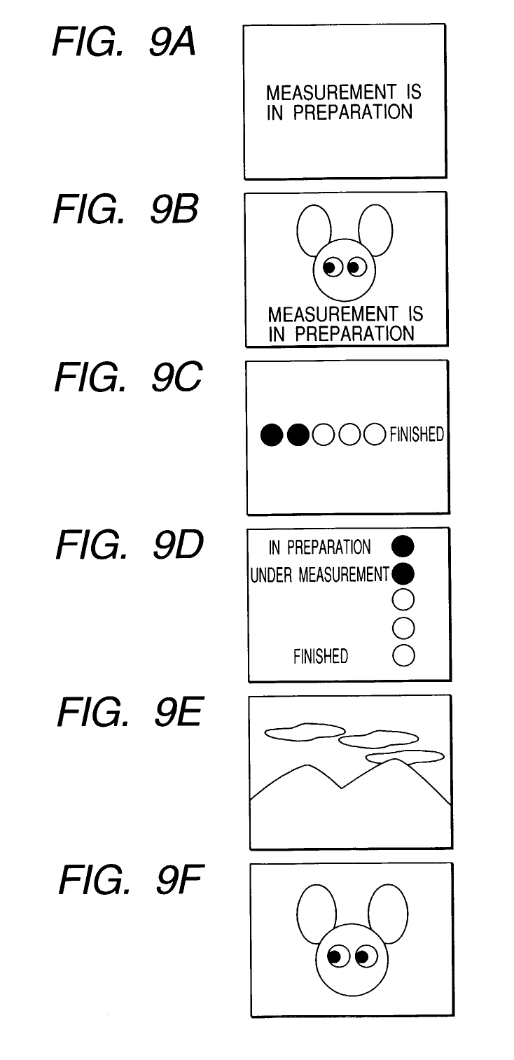

FIGS. 9A to 9F are diagrams showing some examples of a video image shown to the subject to be inspected in the midst of the measurement. These diagrams will now be described.

FIGS. 9A and 9B show examples of a picture for notifying the subject to be inspected that the measurement is being prepared. In the example shown in FIG. 9B, an animation of an animal or the like can be displayed simultaneously with a character string “measurement is in preparation” in order to prevent a child or an infant from being frightened at the inspection. FIGS. 9C and 9D show examples of a picture for notifying the subject to be inspected that the measurement is in preparation, that the measurement is under way, or that the measurement is finished, by using one picture. As the time advances, the content of the scale is changed. FIGS. 9E and 9F show examples of an image, such as a landscape or an animation image of an animal or the like, having no special function. By showing such an image having a movement to the subject to be inspected, the eyes and consciousness of the subject to be inspected can be directed to the screen. Therefore, strain of the inspection can be reduced.

The subject 31 to be inspected watches the picture as an image reflected on the mirror 22 disposed in the shielded room 1. If the picture includes characters, therefore, it is a matter of course that the image displayed on the image projection section 23 has bilaterally inverted characters.

The subject 31 to be inspected can watch the video image projected on the image projection section 23 by directing eyes toward the mirror 22 as described above. Even if the subject 31 to be inspected changes the direction of the neck to watch the video image, therefore, it is possible to prevent the region to be measured in the inspection from being moved. Even if the subject 31 to be inspected watches the image projection section, therefore, it is possible to prevent the measurement from failing because of a shift in the position of the region to be inspected.

The case where an image having some information is projected from the projector 6 onto the image projection section 23 has been described. In the present invention, however, mere projection light having no information may be projected onto the image projection section 23. As a result, the intensity of illumination, which tends to be insufficient, in the shielded room 1 described earlier can be made up for more efficiently.

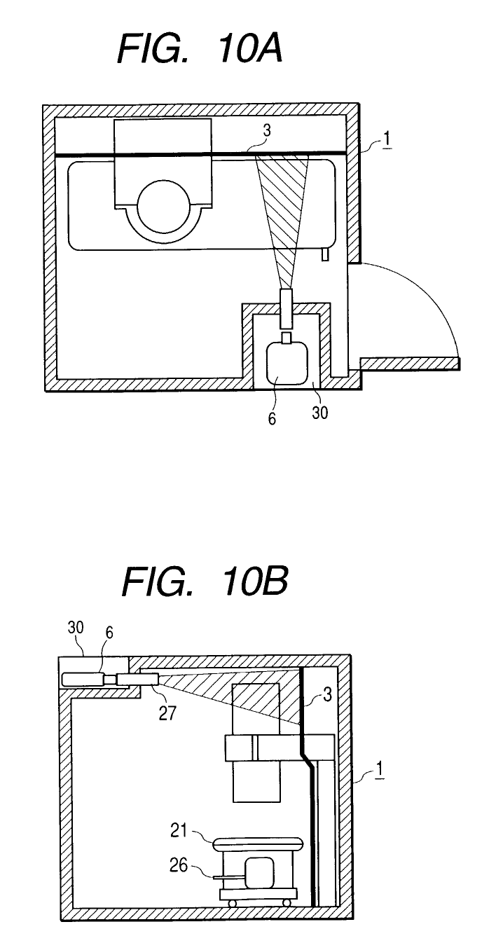

FIGS. 10A and 10B are block diagrams showing a configuration example of a biomagnetic measuring system according to another embodiment of the present invention. FIG. 10A shows a top view, and FIG. 10B shows a side view.

In the embodiment heretofore described, the projector 6 is disposed outside the shielded room 1 that is formed to take the rectangular shape. In the example shown in FIGS. 10A and 10B, a hollow portion 30 is formed in a part of a side wall of the shielded room 1 located directly under the ceiling of the shielded room 1. The projector 6 is disposed within the hollow portion 30. The duct 27 is attached to a wall surface at the back of the hollow portion 30. As a matter of course, the wall surfaces forming the hollow portion 30 are covered by a magnetic material such as permalloy in the same way as other portions. The wall having the hollow portion is a wall opposed to the partition wall 3 having the image projection section 23.

In the embodiment of the present invention having such a configuration, the projector 6 is not seen projecting from the outside of the shielded room 1. The whole shielded room, which is installed in a part of an inspection room or the like, can be made look neat. Furthermore, since there is no projection caused by the projector 6, the whole shielded room can be compact. As a result, transportation and installation of the shielded room can be improved.

According to the embodiments of the present invention, a video image needed by the inspector and a video image to be watched by the subject to be inspected are projected from the projector that is installed outside the shielded room for conducting the biomagnetic measurement, into the shielded room. Therefore, the inspector can use the projected video image in order to position the subject to be inspected. Thus, the inspector need not conduct wasteful actions of going and coming back between the inside and the outside the shielded room many times. Furthermore, it is possible to show a video image for diverting the mind to the subject to be inspected, and the fear and strain of the inspection of the subject to be inspected can be reduced. In addition, according to the embodiments of the present invention, it becomes possible to prevent harmful magnetism from being caused in the shielded room by projecting the video image into the shielded room.

As heretofore described, according to the present invention, it is possible to measure the magnetic field intensity of a living body in an arbitrary measurement location with ease and simple operation, make the inspector conduct the work efficiently, and prevent the subject to be inspected from becoming anxious.

It should be further understood by those skilled in the art that although the foregoing description has been made on embodiments of the invention, the invention is not limited thereto and various changes and modifications may be made without departing from the spirit of the invention and the scope of the appended claims.

What is claimed is:

1. A biomagnetic measuring system for measuring a biomagnetic field, comprising:
an image projection section disposed on a wall surface in a magnetically shielded room; and
a projector for projecting an image onto said image projection section, said projector being disposed outside said magnetically shielded room.

2. The biomagnetic measuring system according to claim 1, wherein said projector is a liquid crystal projector.

3. The biomagnetic measuring system according to claim 1, wherein flux of light from said projector is projected onto said image projection section through a duct disposed so as to pass through a wall surface of said magnetically shielded room.

4. The biomagnetic measuring system according to claim 3, wherein said duct takes a circular shape on an inlet side of an opening disposed outside said magnetically shielded room and takes a square shape on outlet side of said opening disposed inside said magnetically shielded room.

5. The biomagnetic measuring system according to claim 1, wherein a mirror for reflecting the image projected on said image projection section is disposed on a wall surface opposed to the wall surface of said image projection section in said shielded room.

6. The biomagnetic measuring system according to claim 1, wherein at least analysis data is displayed on said image projection section.

7. The biomagnetic measuring system according to claim 1, wherein a progress of an inspection, environmental information, or light having no information is displayed on said image projection section.

8. The biomagnetic measuring system according to claim 1, wherein an operation section for ordering inspection start and inspection end is disposed near said image projection section.
